SEQUENCE LISTING (12 sequences)

SEQ ID NO: 1 - DNA, 41 bp, synthetic construct
  source 1..41: mol_type = other DNA
caaaggactcgcagtcggtccggaggagatgccagatgtga

SEQ ID NO: 2 - DNA, 42 bp, synthetic construct
  source 1..42: mol_type = other DNA
aagtggtggcattgctggttgttttctccattaacgccgcag

SEQ ID NO: 3 - DNA, 21 bp, synthetic construct
  source 1..21: mol_type = other DNA
ggaaaccaaccgcaagacact

SEQ ID NO: 4 - DNA, 23 bp, synthetic construct
  source 1..23: mol_type = other DNA
gacgctgctattgtagcgagttg

SEQ ID NO: 5 - DNA, 20 bp, synthetic construct
  source 1..20: mol_type = other DNA
ttctcccttccgtgtagtcg

SEQ ID NO: 6 - DNA, 20 bp, synthetic construct
  source 1..20: mol_type = other DNA
ggcaaccataccacgaatcg

SEQ ID NO: 7 - DNA, 41 bp, Synthetic construct; identical to SEQ ID NO: 1
  source 1..41: mol_type = other DNA
  modified_base 1: mod_base = OTHER; note = amino C-12

SEQ ID NO: 8 - DNA, 42 bp, synthetic construct; identical to SEQ ID NO: 2
  source 1..42: mol_type = other DNA
  modified_base 1: mod_base = OTHER; note = amino C-12

SEQ ID NO: 9 - DNA, 21 bp, synthetic construct
  source 1..21: mol_type = other DNA
  modified_base 1: mod_base = OTHER; note = amino C-12
ggaaaccaaccgcaagacact

SEQ ID NO: 10 - DNA, 23 bp, synthetic construct
  source 1..23: mol_type = other DNA
  modified_base 1: mod_base = OTHER; note = amino C-12
gacgctgctattgtagcgagttg

SEQ ID NO: 11 - DNA, 22 bp, synthetic construct
  source 1..22: mol_type = other DNA
tattgcacttccgggttagtcc

SEQ ID NO: 12 - DNA, 23 bp, synthetic construct
  source 1..23: mol_type = other DNA
tgttgtacggacactgatacagg

(19) United States
(12) Patent Application Publication
(10) Pub. No.: US 2025/0354206 A1
(43) Pub. Date: Nov. 20, 2025

(54) INTEGRATED SENSING CHIP FOR DETECTING THE PRODUCTS OF LOOP-MEDIATED ISOTHERMAL AMPLIFICATION USING A NON-COLORIMETRIC PROBE-FREE TECHNIQUE

(71) Applicants: The Texas A&M University System, College Station, TX (US); Iowa State University Research Foundation, Inc., Ames, IA (US)

(72) Inventors: Junqi Song, College Station, TX (US); Jinping Zhao, College Station, TX (US); Long Que, Ames, IA (US); Subin Mao, Ames, IA (US)

(21) Appl. No.: 18/970,770

(22) Filed: Dec. 5, 2024

Related U.S. Application Data
(60) Provisional application No. 63/606,470, filed on Dec. 5, 2023.

(51) Int. Cl. C12Q 1/6851; C12Q 1/6853; C12Q 1/6895

(52) U.S. Cl. CPC C12Q 1/6851; C12Q 1/6853; C12Q 1/6895

(57) ABSTRACT

A loop-mediated isothermal amplification (LAMP) platform is disclosed to detect pathogens by integrating LAMP, a rapid tissue lysis method, and an embedded label-free sensor in a reaction/detection chamber, in which the LAMP primers are immobilized on the nanopore thin film. Amplified LAMP products are attached to the sensor surface via the immobilized primers and produce pronounced transducing signals that can be directly measured by an optical spectrometer. The integrated sensing platform requires neither the labeling of LAMP products nor the laborious DNA purification step. This label-free sensing technology enables ultrasensitive, specific, rapid, and cost-effective point-of-care diagnostics of plant, animal, human, and foodborne pathogens.

### CROSS-REFERENCE TO RELATED APPLICATIONS

This application claims the benefit of and priority to U.S. Provisional Application No. 63/606,470, filed on Dec. 5, 2023, which is incorporated by reference herein in its entirety.

### GOVERNMENT SUPPORT

This invention was made with government support under grant no. NA/TEX09675 awarded by the National Institute of Food and Agriculture. The government has certain rights in the invention.

### SEQUENCE LISTING

The instant application contains a Sequence Listing which has been submitted via Patent Center and is hereby incorporated by reference in its entirety. Said .xml copy, created on Aug. 8, 2025, is named “T108205 1160_SL.xml” and is 13,985 bytes in size. The Sequence Listing does not extend beyond the scope of the specification, and does not add new matter.

### TECHNICAL FIELD

The present disclosure generally relates to systems and methods for deoxyribonucleic acid (DNA) amplification and detection. More specifically, the present disclosure relates to systems and methods for fabricating and operating a loop-mediated isothermal amplification (LAMP) platform for the molecular detection of pathogens.

### BACKGROUND

Potato late blight, one of the most economically damaging plant diseases, poses a significant threat to potato production globally. The causal oomycete pathogen, P. infestans, has both asexual and sexual reproduction modes and evolves rapidly to overcome resistant varieties. Under favorable weather conditions, the pathogen can reproduce and spread quickly in the field, and crops can be destroyed within days without any treatments. Once late blight disease is detected during the growing season, continuous weekly spray applications of fungicides are needed to protect crops from further infection and damage. In the United States, the total economic loss caused by late blight is about $210 million plus $77 million of spending on chemical application annually, which averages around $507 per hectare in addition to the standard preventive and control practices for field management.

Detecting the early occurrence of P. infestans infection is essential for effective management of late blight. The development of a rapid point-of-care molecular detection method would allow accurate diagnosis of pathogens and timely decision-making to prevent disease pandemics and reduce disease-caused losses. Multiple molecular detection approaches have been developed over the past several decades. Enzyme-linked immunosorbent assay (ELISA) is an antibody-based approach, which is usually non-species-specific and may cause a high rate of false negatives. Real-time polymerase chain reaction (PCR) and Digital Droplet PCR are species-specific and sensitive, but typically require expensive instruments and reagents as well as experienced personnel.

The development of nucleic acid isothermal amplification methods, such as Loop-mediated isothermal amplification (LAMP), recombinase polymerase amplification (RPA), and rolling circle amplification (RCA), provides on-site applications for diagnosing pathogens. LAMP has been widely used in plant disease detection because of its high sensitivity and specificity. Detection of LAMP products is mainly achieved by direct visualization of the amplified deoxyribonucleic acid (DNA) products labeled with colorimetric dyes or intercalating fluorophores, or by indirect visualization of LAMP byproducts. Several additional methods have been developed to detect the LAMP products, such as a lateral flow device in combination with the antibody-based detection, Raman spectroscopy, and an electrochemical approach.

### SUMMARY

A unique LAMP platform is disclosed herein to detect pathogens by integrating LAMP, a rapid tissue lysis method, and an embedded label-free sensor in a reaction/detection chamber. The LAMP primers are immobilized on a nanopore thin film. Amplified LAMP products are thus attached to the sensor surface via the immobilized primers and produce pronounced transducing signals. These signals can be directly measured by a portable optical spectrometer. The integrated sensing platform requires neither the labeling of LAMP products nor the laborious and time-consuming DNA purification step. This method can be broadly extended to detect diseases in other plant species and to detection of infectious diseases in animals and humans.

Embodiments include systems and methods for the fabrication and operation of a LAMP-based pathogen detection system that is capable of rapid, label-free detection of pathogen DNA at low concentrations. Low concentrations include about 1 femtogram per microliter or at the attomole scale. In certain embodiments, the detection happens in less than one hour. In certain embodiments, the detection happens in about 30 minutes. One such system is a LAMP-based pathogen detection system that includes a LAMP platform having a microfluidic layer bonded to a glass substrate. The glass substrate has an embedded nanopore thin film sensor functionalized with modified LAMP primers. The microfluidic layer defines a sensor-reaction chamber that contains the embedded nanopore thin film sensor. The microfluidic layer is configured to route a LAMP reaction mixture that includes a pathogen sample to the sensor-reaction chamber to form unlabeled LAMP products. The modified LAMP primers of embedded nanopore thin film sensor are configured to bind to a portion of the unlabeled LAMP products for detection. Certain embodiments of the system include an optical spectrometer having an optical probe configured to irradiate the embedded nanopore thin film sensor and measure an optical shift in light reflected by the embedded nanopore thin film sensor to detect the portion of the unlabeled LAMP products bound to the modified LAMP primers of the embedded nanopore thin film sensor.

In some examples, the pathogen sample contains unpurified DNA of a pathogen. In some examples, the LAMP-based pathogen detection system is configured to detect the unpurified DNA of a pathogen in the pathogen sample at concentrations as low as 1 femtogram per microliter (fg/μL). In some examples, the pathogen sample contains crude tissue lysates of plant tissue infected with Phytophthora infestans. In some examples, the unlabeled LAMP products are free of colorimetric dye labels or fluorophore labels. In some examples, the glass substrate of the LAMP platform has a plurality of embedded nanopore thin film sensors, and the microfluidic layer defines a respective sensor-reaction chamber that contains each of the plurality of embedded nanopore thin film sensors. In some examples, the modified LAMP primers contain 5′-amino-C12-modified forward inner primer and 5′-amino-C12-modified backward inner primer. In some examples, the embedded nanopore thin film sensor contains a patterned anodic aluminum oxide (AAO) thin film that is coated with gold and functionalized with the modified LAMP primers. In some examples, the modified LAMP primers are immobilized to the embedded nanopore thin film sensor using at least N-(3-dimethylaminopropyl)-N′-ethylcarbodiimide hydrochloride (EDC)/N-hydroxysuccinimide (NHS) chemistry. In some examples, the LAMP reaction mixture contains an unmodified forward inner primer and an unmodified backward inner primer.

Aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1A is a diagrammatic representation the LAMP platform, according to an embodiment.

FIG. 1B is an enlarged diagrammatic representation of a sensor-reaction chamber of the LAMP platform, according to an embodiment.

FIG. 1C is a photograph of the LAMP platform, according to an embodiment.

FIG. 1D is a scanning electron microscope (SEM) image of an embedded nanopore thin film sensor of a sensor-reaction chamber of the LAMP platform, according to an embodiment.

FIG. 2A is a diagrammatic representation of a process for functionalizing an embedded nanopore thin film sensor of the LAMP platform to immobilize the modified inner primers, followed by LAMP reaction, according to an embodiment.

FIG. 2B is a diagrammatic representation of a process for using a portable optical spectrometer to optically measure transducing signals of LAMP products bound to the embedded nanopore thin film sensor of the LAMP platform, according to an embodiment.

FIG. 2C is a graph illustrating the transducing signal of LAMP products bound to the embedded nanopore thin film sensor of the LAMP platform, according to an embodiment.

FIGS. 3A-3G are a diagrammatic representation of a process for fabricating a glass substrate of the LAMP platform having a nanopore thin film, according to an embodiment.

FIGS. 4A and 4B are representations of LAMP assays performed to detect a particular gene using different amounts of CTAB-extracted genomic DNA from P. infestans, wherein the LAMP products were separated by agarose gel electrophoresis, according to an embodiment.

FIG. 5A is a photograph of the LAMP platform during surface functionalization, according to an embodiment.

FIG. 5B is a photograph of the LAMP platform after surface functionalization, in which the LAMP reaction mixtures are flowed into the sensor-chambers for LAMP reactions, according to an embodiment.

FIG. 5C is a bar graph indicating the measured signals of LAMP products with different ratios of the modified forward inner primer (FIP) to modified backward inner primer (BIP) immobilized on the sensing surface, according to an embodiment.

FIG. 6A is a graph indicating the measured signals of the LAMP products from P. infestans DNA detected using the LAMP platform, according to an embodiment.

FIG. 6B is a bar graph indicating the results of LAMP control experiments, according to an embodiment.

FIG. 6C is a bar graph indicating the measured signals of the LAMP products from different concentrations of P. infestans DNA as part of an experimental determination of the detection limit of the LAMP platform, according to an embodiment.

FIG. 7A is a set of photographs illustrating a P. infestans-infected potato leaf with mild symptoms (right) and a non-infected control (left), according to an embodiment.

FIG. 7B is a bar graph indicating the LAMP platform detection of P. infestans using plant tissue lysates, according to an embodiment.

### DETAILED DESCRIPTION

The present disclosure describes various embodiments related to systems and methods for a LAMP platform. In certain embodiments, the platform is manufactured to detect pathogens by integrating LAMP, a rapid tissue lysis method, and an embedded label-free sensor in a reaction/detection chamber, in which the LAMP primers are immobilized on the nanopore thin film. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “plurality” as used herein refers to two or more items or components. The terms “about” or “approximately” are defined as being close to as understood by one of ordinary skill in the art. In one non-limiting embodiment, these terms are defined to be within 10%, preferably within 5%, more preferably within 1%, and most preferably within 0.5%.

The terms “removing,” “removed,” “reducing,” “reduced,” or any variation thereof, when used in the claims and/or the specification includes any measurable decrease of one or more components in a mixture to achieve a desired result. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The terms “wt. %”, “vol. %”, or “mol. %” refers to a weight, volume, or molar percentage of a component, respectively, based on the total weight, the total volume of material, or total moles, which includes the component. In a non-limiting example, 10 grams of component in 100 grams of the material is 10 wt. % of component.

LAMP is a nucleic acid amplification technique and has been widely used for detection of pathogens in many organisms. Current LAMP-based sensors usually require LAMP products to be labeled (for example, using colorimetric dyes or intercalating fluorophores) in order for the products to be detected. To address this shortcoming, a novel label-free LAMP platform was developed that includes at least one nanopore thin-film sensor embedded inside a LAMP reaction chamber. Modified LAMP primers are immobilized on the surface of the sensor, allowing LAMP products to be synthesized and attached to the sensor surface via the immobilized primers. After the LAMP reaction components are rinsed out of the reaction chamber, the amplified LAMP products attached to the sensor surface can be measured by a portable optical spectrometer through an optical fiber probe, giving rise to significantly increased transducing signals. In an example, an embodiment of the LAMP platform was used to detect the causal agent of late blight, Phytophthora infestans (P. infestans), which is one of the most devastating plant pathogens and poses a major threat to sustainable crop production worldwide. The LAMP platform can detect trace levels of pathogen DNA as low as 1 femtogram per microliter (fg/μL) in 30 minutes (min), which is at least 10 times more sensitive than the currently available methods to detect LAMP products. This label-free sensing technology provides for ultra-sensitive, highly specific, rapid, and cost-effective pathogen detection suitable for the point-of-care testing. The disclosed embodiments of the LAMP platform can also be broadly applied for on-site diagnostics of plant, animal, human, and foodborne pathogens, such as such as COVID-19, avian flu, swine flu, and foodborne pathogenic bacteria.

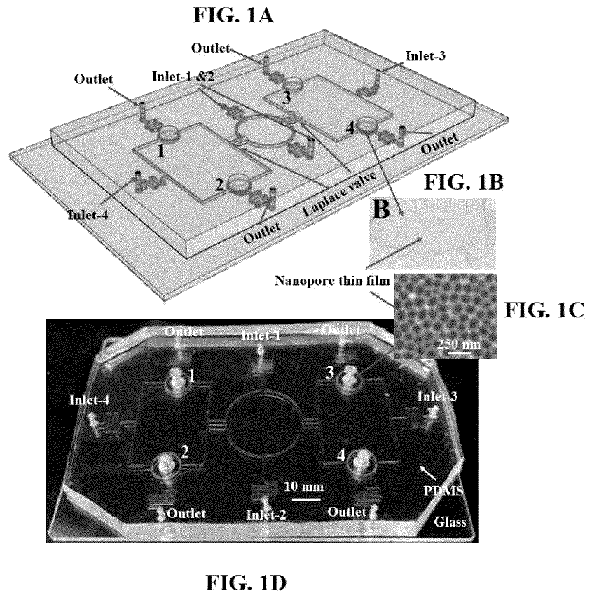

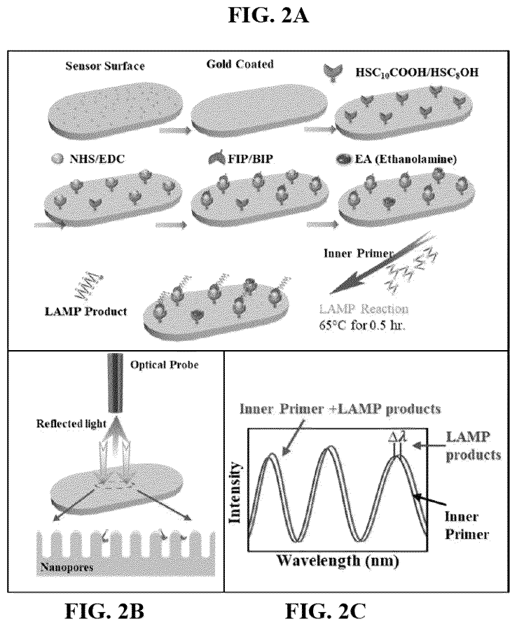

FIG. 1A illustrates an example embodiment of the LAMP platform. For the illustrated embodiment, the LAMP platform includes four sensor-reaction chambers (labeled as 1, 2, 3, and 4 in FIG. 1A) with a respective embedded nanopore thin film sensor disposed at the bottom of each of the sensor-reaction chambers. An enlarged schematic view of a sensor-reaction chamber of the LAMP platform is illustrated in FIG. 1B. A scanning electron microscope (SEM) image of an embedded nanopore thin film sensor is illustrated in FIG. 1D. The embedded nanopore thin film serves as the label-free sensor to monitor LAMP products. A picture of a fabricated microfluidic device for an embodiment of the LAMP platform is shown in FIG. 1C. During manufacture of the LAMP platform, the surface of each embedded nanopore thin film sensor is functionalized with inner primers by an EDC/NHS chemistry monolayer. During operation of the LAMP platform, the chemicals for the LAMP reaction are flowed through microfluidic channels into the sensor-reaction chambers. The surface functionalization procedure and LAMP products are shown in FIGS. 2A-2B, as discussed below. After incubation at 65 degrees Celsius (° C.) for 30 min (or as indicated), the sensor-reaction chambers are then rinsed by flowing saline-sodium citrate (SSC), phosphate-buffered saline (PBS), and double-distilled water (ddH₂O). The transducing signals (optical signal shift (AA)) of the LAMP products are then measured by a portable optical spectrometer, as illustrated FIGS. 2B and 2C. In some embodiments, the combination of the LAMP platform and the spectrometer may be referred to herein as a LAMP-based pathogen detection system. In some implementations, the nanopore thin film based optical sensor can also be replaced by nanoelectronic sensors enabled by one-dimensional and two-dimensional nanomaterials or transistors.

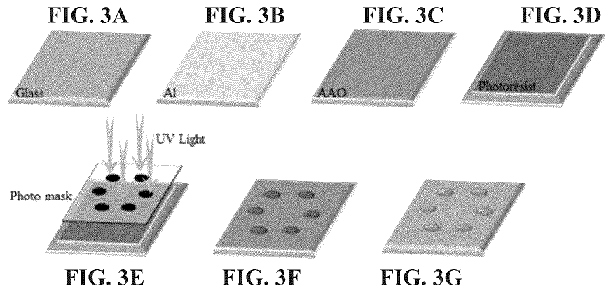

To manufacture the LAMP platform, a glass substate is fabricated having a nanopore thin film, which forms the embedded nanopore thin film sensor of the sensor-reaction chambers after functionalization. In some embodiments, the glass substate with the nanopore thin film is fabricated in accordance with the fabrication process set forth in the 2018 journal article entitled, “Rapid multiplexed detection of beta-amyloid and total-tau as biomarkers for Alzheimer's disease in cerebrospinal fluid,” by Song, Chao, Pan Deng, and Long Que (Nanomedicine: Nanotechnology, Biology and Medicine, 14:1845-52). An example illustration of this fabrication process is illustrated in FIGS. 3A through 3G. As illustrated in FIG. 3A, the process begins with a clean (for example, oxygen-plasma treated) glass substate. Next, as illustrated in FIG. 3B, an aluminum (Al) thin film is deposited on the glass substrate using chromium (Cr) as an adhesion layer. As illustrated in FIG. 3C, the Al thin film is converted to anodic aluminum oxide (AAO) using a two-step anodization process. As illustrated in FIG. 3D, a photoresist is subsequently applied to the surface of the AAO thin film. The photoresist is then patterned using optical lithography, as illustrated in FIG. 3E, to form a mask that covers and protects portions of the surface, as illustrated in FIG. 3F. Subsequently, a time-controlled, wet-etching processes is performed to remove the AAO thin film from the exposed portions of the surface, and then the mask is removed to form the glass substrate of FIG. 3G having a patterned AAO thin film. Then, a layer of gold (Au) having a thickness from about 1 nm to about 2 nm is deposited on the patterned AAO thin film using a layer of chromium (Cr) having a thickness of about 0.5 nm as the adhesion layer, yielding the unfunctionalized, patterned thin film.

Additionally, to manufacture the LAMP platform, a microfluidic layer is fabricated to be positioned on top of the fabricated glass substrate. In an example embodiment, the microfluidic layer is fabricated by first washing a 4-inch silicon wafer with acetone, isopropanol, and DI water sequentially. Subsequently, a negative photoresist (for example, SU-8) is dispensed onto the washed silicon wafer, followed by optical lithography. In an embodiment, the negative photoresist is dispensed onto the washed silicon wafer spinning at a speed of 3000 revolutions per minute (rpm) for 60 seconds (secs). As a result, an approximately 80 micrometer (μm) thick photoresist mold is formed. A PDMS replication from the photoresist mold is then formed by curing liquid PDMS polymer. In an embodiment, the curing of the liquid PDMS polymer takes about 2 hours at 65° C. The inlets and outlets of the LAMP platform (as illustrated in FIG. 1A) are formed in the PDMS layer using a biopsy punch, followed by an oxygen plasma treatment, to form the microfluidic layer of the LAMP platform. Finally, the PDMS microfluidic layer is bonded with fabricated glass substrate having a number of nanopore thin film sensors, forming the LAMP platform.

After bonding the fabricated microfluidic layer to the fabricated glass substrate, the patterned thin film illustrated in FIG. 3G is functionalized to form the embedded nanopore thin film sensors of the sensor-reaction chambers of the LAMP platform. In an example embodiment, 5′-amino-C12-modified inner primers, including forward inner primer (FIP) and backward inner primer (BIP), are immobilized on the surface of the patterned thin film of the glass substrate using EDC/NHS chemistry. The functionalization steps are schematically illustrated in FIG. 2A. Briefly, a solution containing HSC₁₀COOH and HSC₈OH is applied to the surface of the fabricated glass substrate and incubated, followed by a rigorous wash with ethanol. After the surface was dried, a solution containing NHS and EDC is applied to the surface and incubated, followed by a rinse. Next, the primers are functionalized on the sensor surface by immersing the sensor into the inner primer solution containing modified FIP and BIP primers. To remove non-specifically adsorbed chemicals, ethanolamine (EA) is applied to the surface to react with the non-occupied sites activated by the EDC/NHS, followed by a rinse, forming the embedded nanopore thin films of the sensor-reaction chambers of the LAMP platform.

For example, a solution of 0.1 millimolar (mM) (1:9) HSC₁₀COOH/HSC₈OH is applied to the surface of the fabricated glass substrate and incubated for 30 min, followed by a rigorous wash with ethanol. After the surface was dried naturally, a solution containing 0.2 molar (M) NHS and 0.05 M EDC is applied to the surface and incubated for 30 min, followed by a rinse with a PBS buffer. Next, the primers are functionalized on the sensor surface by immersing the sensor into the inner primer solution containing modified FIP and BIP primers (for example, amino-C12-modified at the 5′ end) each at a 1.6 micromolar (μM) concentration or as indicated. To remove non-specifically adsorbed chemicals, 100 μL of 1 M ethanolamine (EA) is applied to the surface to react with the non-occupied sites activated by the EDC/NHS, followed by a rinse with the PBS buffer, forming the embedded nanopore thin films of the sensor-reaction chambers of the LAMP platform.

Materials for device fabrication discussed herein include: polydimethylsiloxane (PDMS) and its curing agent purchased from Dow Corning, Inc., SU-8 photoresist purchased from MicroChem, Inc., aluminum targets of high purity (99.99%) purchased from Kurt J. Lesker, Inc., and deionized (DI) water was made by tap water through a DI water purification system (Millipore, USA). Chemicals discussed herein for sensing surface functionalization include: 11-mercaptoundecanoic acid (HSC₁₀COOH, 99% purity), 8-mercapto-1-octanol (HSC₈OH, 98% purity), N-(3-dimethylaminopropyl)-N′-ethylcarbodiimide hydrochloride (EDC), N-hydroxysuccinimide (NHS), and ethanolamine (EA), all of which were purchased from Sigma-Aldrich.

In order to evaluate the performance of the LAMP platform, the platform was used to detect the causal agent of late blight, Phytophthora infestans. To simplify the LAMP platform and reduce the time required for point-of-care detection of pathogens, a preparation method was applied to prepare crude tissue lysates that directly served as templates for LAMP. DNA from the P. infestans mycelia was extracted. LAMP primers targeting the P. infestans internal transcribed spacer (ITS) gene were (the modified FIP and BIP primers) were immobilized on the embedded nanopore thin film of the sensor-reaction chambers. LAMP reaction was conducted using the unmodified primers. The resulting LAMP products are bound to the sensor via immobilized modified inner primers, whereas substantially all unbound DNA, primers, polymerase, dNTPs, and chemicals are removed by subsequent washing before the optical measurements were performed. As the LAMP products are bound to the sensor, aerosols that typically cause contamination are not generated. Therefore, the systems and methods disclosed herein can significantly reduce or eliminate carry-over contamination.

In an example, the P. infestans strain US-23 was cultured on a rye A medium at 17° C. for two weeks. Sporangia were collected with chilled water, and the concentration of sporangia was measured and adjusted to 15,000 sporangia per milliliter. Droplets of 10 μL were applied to the abaxial sides of potato leaves. Infected potato plants were incubated in a moist chamber with over 90% relative humidity at 18° C. In an example, potato tissues of approximately 4 square millimeters (mm²) were collected into a 1.7 mL tube containing 200 μL of 0.5 N NaOH and incubated at 65° C. for 5 min. Ten microliters of the lysed tissues was transferred to another tube and mixed with 400 μL Tris-HCl (pH 7.0). The neutralized tissue lysates were diluted 20 times with ddH₂O and subjected to LAMP assays.

To extract DNA from P. infestans, fresh mycelia were collected and grounded in liquid nitrogen with mortar and pestle and homogenized in the extraction buffer (for example, 0.2 M Tris-HCl, 0.25 M NaCl, 25 mM EDTA, 0.5% SDS, pH 8.0). The lysates were transferred into 1.7 mL centrifuge tubes, and RNA was removed by adding RNase A at a concentration of 20 microgram per milliliter (μg/mL) and incubation at 65° C. for 15-30 min in a water bath. After cooling down to room temperature, one-third volume of 3 M NaOAc (pH 5.2) and an equal volume of isopropanol was added into the samples, followed by incubation at −20° C. for 20 min and centrifugation at 14,000×g for 10 min. The supernatant was aspirated, and the pellet was washed with 70% ethanol twice. After drying out at room temperature, the pellet was dissolved in TE (for example, 10 mM Tris-HCl, 1 mM EDTA, pH 7.5). After further purification by phenol/chloroform/isoamyl, the DNAsamples were quantified by the NanoDrop™ OneC Microvolume UV-Vis Spectrophotometer (Thermo Fisher, USA). As such, it may be appreciated that no laborious DNA purification or isolation techniques are needed to prepare a pathogen sample for LAMP analysis.

The LAMP primers targeting the P. infestans internal transcribed spacer (ITS) gene were designed using the PrimerExplorer tool V5 edition (http://primerexplorer.jp/lampv5e/index.html). The standard unmodified LAMP primers were synthesized by Eurofins Genomics LLC (USA). The 5′-amino-C12-modified FIP and BIP primers were synthesized by Integrated DNA Technologies (USA). The primer sequences for the ITS gene are listed in Table S1. Real-time PCR primers targeting the same P. infestans ITS gene region were designed using the Primer3Plus tool (https://www.primer3plus.com/). Primers were synthesized by Eurofins Genomics LLC (USA), and their sequences are listed in Table 1.

TABLE 1
The LAMP and PCR primer sequences for the P. infestans ITS gene.

| Primer | SEQ | | Length | |
|---|---|---|---|---|
| Number | ID NO: | Sequence | (nt) | Annotation |
| Lamp47 | 1 | CAAAGGACTCGCAGTCGGTCCGGAGG | 39 | FIP for P. infestans ITS |
| | | AGATGCCAGATGTGA | | gene |
| Lamp48 | 2 | AAGTGGTGGCATTGCTGGTTGTTTTCTC | 40 | BIP for P. infestans ITS |
| | | CATTAACGCCGCAG | | gene |
| Lamp49 | 3 | GGAAACCAACCGCAAGACACT | 20 | LoopF for P. infestans ITS |
| | | | | gene |
| Lamp50 | 4 | GACGCTGCTATTGTAGCGAGTTG | 20 | LoopB for P. infestans ITS |
| | | | | gene |
| Lamp51 | 5 | TTCTCCCTTCCGTGTAGTCG | 20 | F3 for P. infestans ITS |
| | | | | gene |
| Lamp52 | 6 | GGCAACCATACCACGAATCG | 18 | B3 for P. infestans ITS |
| | | | | gene |
| Lamp53 | 7 | AMC12- | 39 | FIP for P. infestans ITS |
| | | CAAAGGACTCGCAGTCGGTCCGGAGG | | gene with Amino C-12 at |
| | | AGATGCCAGATGTGA | | 5′ |
| Lamp54 | 8 | AMC12- | 40 | BIP for P. infestans ITS |
| | | AAGTGGTGGCATTGCTGGTTGTTTTCTC | | gene with Amino C-12 at |
| | | CATTAACGCCGCAG | | 5′ |
| Lamp55 | 9 | AMC12- | 20 | LoopF for P. infestans ITS |
| | | GGAAACCAACCGCAAGACACT | | gene with Amino C-12 at 5′ |
| Lamp56 | 10 | AMC12- | 20 | LoopB for P. infestans ITS |
| | | GACGCTGCTATTGTAGCGAGTTG | | gene with Amino C-12 at 5′ |
| ojp2715 | 11 | TATTGCACTTCCGGGTTAGTCC | 22 | Real-time PCR forward |
| | | | | primer for P. infestans |
| | | | | ITS gene |
| ojp2716 | 12 | TGTTGTACGGACACTGATACAGG | 23 | Real-time PCR reverse |
| | | | | primer for P. infestans ITS |
| | | | | gene |

In an example, to perform the LAMP reaction, 2.5 μL of the Isothermal Amplification Buffer (10×), 1.5 μL of the MgSO₄ (100 mM), 0.875 μL of the dNTP Mix (10 mM Each), 1 μL of each FIP and BIP Primer (40 μM), 1 μL of each F3 and B3 Primer (5 M), 1 μL of each LoopF and LoopB Primer (10 μM), 1 μL of NEB Bst 2.0 WarmStart® DNA Polymerase (USA), and 1 μL of the DNA sample with indicated concentrations were mixed on ice and adjusted to a total volume of 25 μL with ddH₂O. LAMP was performed at 65° C. for 30 min or as indicated. To monitor LAMP products by electrophoresis, the LAMP products were run on an agarose gel (1.2%), followed by staining with ethidium bromide and visualized with UV light using an Azure 200 Imaging System (Azure Biosystems, USA).

To set up the LAMP reaction on the LAMP platform, the modified FIP and BIP primers were immobilized on the embedded nanopore thin film of the sensor-reaction chambers, as discussed above. In an example, the unmodified FIP and BIP primers were added to the LAMP reaction mixture at different ratios as follows: for the ratio of 1:1, 1.6 micromoles per liter (μmol/L) of each modified FIP and BIP primers was immobilized, and 1.6 μmol/L of each unmodified FIP and BIP primers was added to the LAMP reaction mixture; for the ratio of 3:1, 2.4 μmol/L of each modified primers was immobilized, and 0.8 μmol/L of each unmodified primers was added to the LAMP reaction mixture; for the ratio of 1:3, 0.8 μmol/L of each modified primers was immobilized, and 2.4 μmol/L of each unmodified primers was added to the LAMP reaction mixture. The LAMP reactions were performed at 65° C. for 30 min or as indicated.

For the measurements, an optical fiber probe (Ocean Optics, Inc.) delivered white light perpendicularly to the surface of the nanopore thin film sensor, the reflected light was collected by the same optical fiber probe, leading to a miniature spectrometer (Ocean Optics, Inc.), as illustrated in FIGS. 2B and 2C. First, a reference optical signal was obtained from a fresh sensor (for example, the patterned thin film) without any surface chemical functionalization. Afterwards, a series of optical signals from the sensor surface were obtained, as illustrated in FIG. 2B, after each step of surface chemical functionalization until the inner primers were functionalized on the sensor surface, as illustrated in FIG. 2A. Then, a sensor-reaction chamber was loaded with LAMP reaction mixture, followed by an incubation at 65° C. for 30 min or as indicated. After LAMP reactions were complete, the sensor-reaction chamber was rigorously rinsed several times before the optical measurements were performed.

For comparison, real-time PCR was performed using a series of concentrations of P. infestans genomic DNA extracted by the cetyltrimethylammonium bromide (CTAB) method as templates. In an example, for each reaction, 0.1 μL of each forward and reverse primer (40 μM), 5 μL of KAPA SYBR FAST qPCR Master Mix (ROX Low, Roche, USA), and 0.1 μL of DNA sample with indicated concentrations were mixed on ice and adjusted to a total volume of 10 μL with ddH2O. PCR was conducted on a QuantStudio 6 Flex Real-Time PCR System (Thermo Fisher, USA) under the following conditions: 95° C. for 3 min and 40 cycles of 95° C. for 3 sec and 65° C. for 30 sec. The melting curve analysis was performed immediately after real-time PCR under the following conditions: 95° C. for 15 sec, 65° C. for 1 min, a gradual increase to 95° C. at a ramp of 0.05° C./sec, and 95° C. for 15 sec. Each real-time PCR was repeated 3 times. The calibration curve was generated by using a series of diluted P. infestans DNA of known concentrations. The quantification cycle (Cq) values were plotted using Python3.

After collecting the optical signals from the nanopore thin film sensor before and after the LAMP reaction, then averaging the shifts of the peaks of the interference fringes of the optical signals, the transducing signals (i.e., optical signal shift) from the sensor were obtained. It may be appreciated that the control experiments may cause some level of spectral shift in the optical signal, likely due to nonspecific binding of the reaction constituents to the sensor surface, the occasional extension of immobilized primers, and the unspecific association of the immobilized primers to DNA templates and reacting primers. The shift was considered as a background shift, which was subtracted from all the measured results. All optical measurement data is presented in mean plus-or-minus standard deviation (+SD). Independent experiments were performed at least three times.

LAMP requires 4 or 6 specific primers (for example, outer and inner primers with or without loop primers), such as forward primer F3, backward primer B3, forward inner primer FIP, backward inner primer BIP, forward loop primer LoopF, and backward loop primer LoopB. To evaluate the performance of the LAMP platform, six LAMP primers were designed based on the ITS gene sequence of P. infestans using the PrimerExplorer V5 software. To determine the effectiveness of the designed LAMP primers, LAMP reactions were performed using a series concentration of P. infestans DNA as templates. The LAMP reactions with 30- and 40-min incubation times produced specific amplification from as low as 500 fg/μL DNA template, which coincided with the detection limits of LAMP reaction in the previous studies.

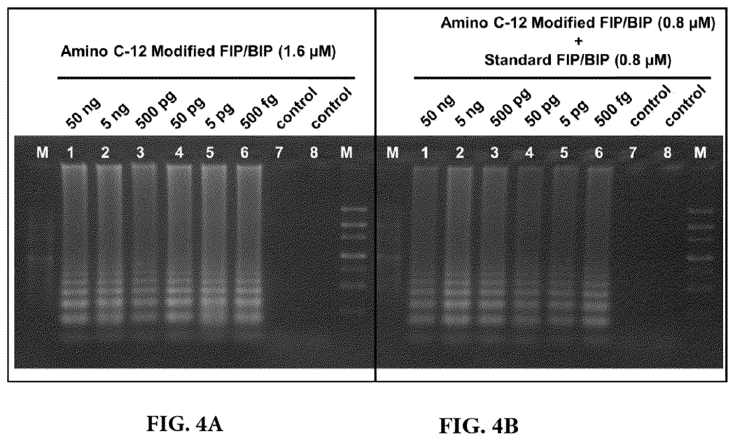

As part of investigations into integrating LAMP with the nanopore thin film sensors of the LAMP platform, experiments were conducted to evaluate whether the amino-C12-modified primers, which were designed to be functionalized on the sensors, are suitable for LAMP amplification. In an example, for this evaluation, LAMP reactions were conducted using different combinations of 5′-amino-C12-modified and standard unmodified primers. More specifically, LAMP assays were performed for 40 min to detect the ITS gene using different amounts (500 fg to 50 ng) of CTAB-extracted genomic DNA from P. infestans as template, and LAMP products were separated by agarose gel electrophoresis in Lanes 1 to 6, as illustrated in FIG. 4. In FIG. 4, Lanes 7 and 8 were negative controls using water as a template, and M indicates a DNA ladder marker. As indicated by the results illustrated in FIG. 4, the modified BIP/FIP primers showed similar LAMP amplifications compared with unmodified primers, indicating that immobilized BIP/FIP primers are suitable for LAMP. The specificity and reliability of these modified primers for LAMP amplification were demonstrated with a series of concentrations of genomic DNA extracted from P. infestans using the traditional CTAB method, as LAMP products were only amplified from the pathogen genomic DNA but not from water.

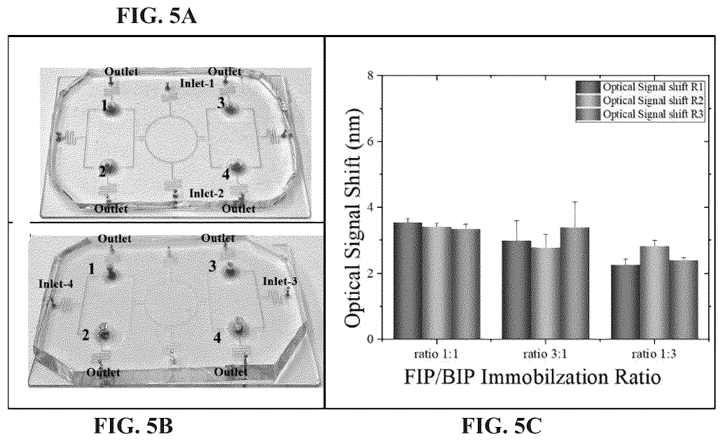

The functions of the LAMP platform have been demonstrated using the food dyes to mimic the chemicals for visibility. As shown in FIG. 5A, using the Inlet-1 and Inlet-2 to flow the chemicals (mimicked by green dye), the four nanopore thin film sensors (1, 2, 3, and 4) were functionalized step-by-step, as illustrated in FIG. 2A. After functionalization, as illustrated in FIG. 5B, LAMP reaction mixtures (mimicked by blue dye) were introduced to the nanopore thin film sensors 1 and 2 by flowing through the Inlet-3. Nanopore thin film sensors 3 and 4 were used for the control experiments, and LAMP reagents (mimicked by red dye) were provided by flowing through the Inlet-4. Therefore, LAMP reaction and detection were performed independently in the chambers ½ and ¾. After LAMP reactions, all four chambers were rinsed rigorously by flowing 2×SSC and PBS before the measurements of the optical signals from the nanopore thin film sensors disposed within each sensor-reaction chamber.

As noted herein, to achieve label-free detection of pathogens using the LAMP platform, a portion of the modified inner primers are immobilized on the nanopore thin film sensors, as shown in FIG. 2A, such that the resulting LAMP products are bound to the sensor via immobilized modified inner primers, whereas substantially all unbound DNA, primers, polymerase, dNTPs, and chemicals are removed by subsequent washing. In an example, to enable immobilization, FIP and BIP were modified by incorporating the amino-C12 modifier to provide a 12-carbon spacer onto the 5′ terminus of each inner primer. For each LAMP reaction, 20 μL of the 5′-amino C12-modified FIP and BIP each at a concentration of 1.6 μM was immobilized on the nanopore thin film sensors. After washing with PBS and ddH₂O, the LAMP reaction was set up by mixing 2.5 μL of 10×LAMP reaction buffer, 1.5 μL of 100 mM MgSO₄, 1 μL of 40 mM dNTPs, 1 μL each of 40 μM FIP and BIP, 1 μL each of 5 μM F3 and B3, 1 μL each of 10 μM LoopF and LoopB, 1 μL of Bst 2.0 Warmstart DNA polymerase (8,000 U/mL, New England Biolabs), DNA sample, and ddH₂O in a total volume of 25 μL and incubated at 65° C. for 30 min or as indicated. After briefly washing with 2×SSC at 65° C. and PBS at room temperature, the bound LAMP products were measured by a spectrometer.

To identify the optimal amount of the modified inner primers (FIP/BIP) to be immobilized on the sensing surface, different ratios of immobilized versus non-immobilized FIP/BIP primers were examined. As shown in FIG. 5C, among the three combinations of 1:3, 3:1, and 1:1, the 1:1 ratio produced more consistent and reliable transducing signals (i.e., the shift of the interference fringes) after the LAMP reactions. Hence, the 1:1 ratio of the FIP/BIP primers was used for the rest of the experiments.

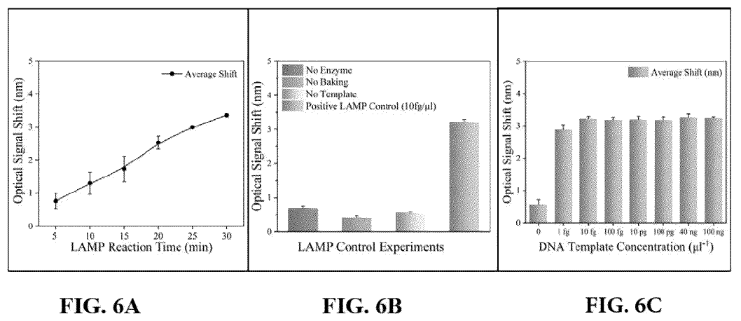

Using the LAMP platform, LAMP was performed under different incubation time from 5 to 30 min using 10 femtograms (fg) of the genomic DNA isolated from P. infestans as a template and the transducing signals were measured. As expected, the yields of LAMP products were positively correlated with reaction time, resulting in the increased transducing signals. Specifically, the shift of the interference fringes of the optical signal was approximately 1.25 nanometers (nm) at 10 min and reached to approximately 3.25 nm at 30 min, as illustrated in FIG. 6A. To validate the label-free detection approach, several additional control experiments were conducted under the following conditions: (1) LAMP without the Bst 2.0 WarmStart DNA Polymerase; (2) LAMP without incubation at 65° C.; and (3) LAMP without template DNA. The results of these control experiments are illustrated in FIG. 6B. For all these cases, the transducing signals (0.62±0.09 nm) are much smaller than those (3.23±0.07 nm) of the standard LAMP reactions, indicating that the disclosed LAMP platform can successfully detect the LAMP products. For pathogen detection using the nanopore thin film sensors of the LAMP platform, the cut-off value is defined as the mean value of the average of transducing signals from LAMP using 100 fg/μL of purified pathogen DNA as a template (Δλ_{100 fg/μl}=3.17 nm) and the average of negative control readings (Δλ_{control}=0.57 nm), and it is calculated to be 1.87 nm.

The detection limit of LAMP platform was experimentally evaluated using a serial dilution of P. infestans DNA as templates under the fixed reaction time of 30 min. As shown in FIG. 6C, as low as 1 fg/μL of template DNA can be readily detected with a transducing signal of 2.89±0.14 nm in 30 min, which is 54.5% above the cut-off value. To compare the detection limit of LAMP platform with conventional real-time PCR, real-time PCR was performed using the same template DNA samples. Consistent with previous studies, the SYBR Green real-time PCR can detect as low as 1 picograms per microliter (pg/μL) of template DNA, which is within the linear amplification range with optimal efficiency parameters. Therefore, the disclosed LAMP platform assay demonstrated a substantially higher sensitivity that is 1000 times more sensitive than SYBR Green real-time PCR. The detection limit of the disclosed label-free LAMP platform is at least 10 times more sensitive than the currently best reported method, which can detect 10 fg/μL, while conventional LAMP has been reported to only detect about 1 pg/uL of target sequences from certain pathogens within 30 min. With further optimization of the nanopore thin film sensor, primer modification, and the amount of immobilized LAMP primers, the detection limit can be further improved to be lower than 1 fg/μL. It may be noted that a detection limit of 1 fg/μL corresponds to an attomolar level of 1.6×10⁻⁶ attomoles per microliter.

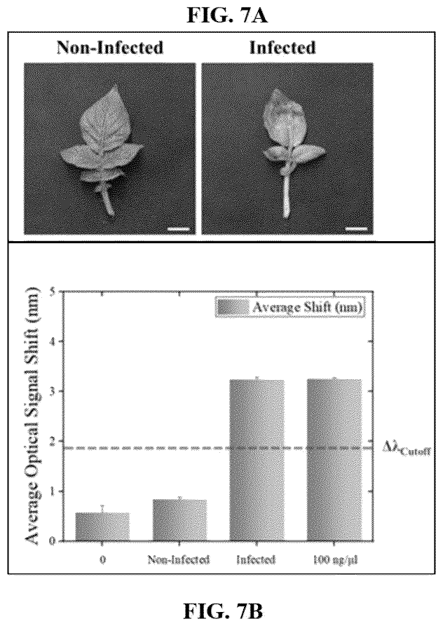

To validate the feasibility of using the LAMP platform for detection of pathogens from infected plants, assays were performed with the LAMP platform using the tissue lysis samples prepared from P. infestans-infected potato plants with mild symptoms during the early stage of infection, as illustrated in FIG. 7A. The results of these assays are illustrated by FIG. 7B. In an example, the transducing signal is 3.24±0.05 nm, which is 73.2% above the set cut-off value of 1.87 nm, and is comparable to that (3.25±0.02 nm) obtained by the LAMP platform using the CTAB-purified pathogen DNA as a template. By contrast, the negative controls without template DNA or using the tissue lysates prepared from non-infected plants only gave rise to 0.57±0.14 nm and 0.84±0.05 nm, respectively, both of which are noticeably below the cut-off point, as indicated in FIG. 7B. P. infestans from the potato plants with mild symptoms was also detected using a standard LAMP reaction (40 min), which validates the results of the LAMP platform assays. These data show that plant tissue lysates are suitable to serve as LAMP templates and the rapid tissue lysis method significantly simplifies and reduces the time for point-of-care detection. Taken together, the results demonstrate the specificity and applicability of the LAMP platform for efficient detection of plant pathogens.

As discussed herein, the LAMP platform offers a new portable platform to detect pathogens using label-free sensors with ultra-sensitivity (approximately 1 fg/mL) and high specificity in a short period of time (approximately 25 min), indicating its suitability for point-of-care testing and on-site diagnostics. Unlike the currently available LAMP-based sensors, which usually require labeling of LAMP amplicons that adds additional time, cost, and complexity, the label-free LAMP chip is capable of directly detecting LAMP products bound to the nanopore thin film sensors. The oomycete pathogen P. infestans produces sporangia, specialized receptacles in which asexual spores are formed and spread by wind or water. Late blight forecast largely depends on detection and monitoring of aerial sporangia. In combination with the air filter collection sample devices, the disclosed LAMP platform may also provide a solution for timely and accurate forecast of P. infestans in the field. Furthermore, this LAMP platform provides a readily adaptable and generic technical platform for detecting a variety of nucleic acid products. It can be operated in combination with RT-LAMP and RCA for detection of both DNA and RNA molecules. Therefore, the disclosed label-free LAMP platform is ultra-sensitive, highly specific, rapid, and cost-effective and can be broadly applied to detect a wide range of diseases in plants, animals, and humans. Furthermore, the systems and methods disclosed herein can significantly reduce or eliminate carry-over contamination. The LAMP products are bound to the sensor, which prevents the generation of aerosols that typically cause contamination.

Other objects, features, and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A loop-mediated isothermal amplification (LAMP)-based pathogen detection system, comprising:
a LAMP platform with a microfluidic layer bonded to a glass substrate, the glass substrate having an embedded nanopore thin film sensor functionalized with modified LAMP primers, and the microfluidic layer defining a sensor-reaction chamber that contains the embedded nanopore thin film sensor, the microfluidic layer being configured to route a LAMP reaction mixture containing a pathogen sample to the sensor-reaction chamber to form unlabeled LAMP products, the modified LAMP primers of embedded nanopore thin film sensor being configured to bind to a portion of the unlabeled LAMP products for detection; and
an optical spectrometer having an optical probe configured to irradiate the embedded nanopore thin film sensor and measure an optical shift in light reflected by the embedded nanopore thin film sensor to detect the portion of the unlabeled LAMP products bound to the modified LAMP primers of the embedded nanopore thin film sensor.

2. The LAMP-based pathogen detection system of claim 1, wherein the pathogen sample contains unpurified deoxyribonucleic acid (DNA) of a pathogen.

3. The LAMP-based pathogen detection system of claim 2, wherein the LAMP-based pathogen detection system is configured to detect the unpurified DNA of the pathogen in the pathogen sample at concentrations as low as 1 femtogram per microliter (fg/μL).

4. The LAMP-based pathogen detection system of claim 1, wherein the pathogen sample contains crude tissue lysates of plant tissue infected with Phytophthora infestans.

5. The LAMP-based pathogen detection system of claim 1, wherein the unlabeled LAMP products are free of colorimetric dye labels or fluorophore labels.

6. The LAMP-based pathogen detection system of claim 1, wherein the glass substrate of the LAMP platform has a plurality of embedded nanopore thin film sensors, and the microfluidic layer defines a respective sensor-reaction chamber containing each of the plurality of embedded nanopore thin film sensors.

7. The LAMP-based pathogen detection system of claim 1, wherein the modified LAMP primers contain 5′-amino-C12-modified forward inner primer and 5′-amino-C12-modified backward inner primer.

8. The LAMP-based pathogen detection system of claim 1, wherein the embedded nanopore thin film sensor contains a patterned anodic aluminum oxide thin film coated with gold and functionalized with the modified LAMP primers.

9. The LAMP-based pathogen detection system of claim 1, wherein the modified LAMP primers are immobilized to the embedded nanopore thin film sensor using at least N-(3-dimethylaminopropyl)-N′-ethylcarbodiimide hydrochloride (EDC)/N-hydroxysuccinimide (NHS) chemistry.

10. The LAMP-based pathogen detection system of claim 1, wherein the LAMP reaction mixture contains unmodified forward inner primer and unmodified backward inner primer.
